(19) United States
(12) Patent Application Publication
(10) Pub. No.: US 2018/0218115 A1
(43) Pub. Date: Aug. 2, 2018

(54) DISEASE RISK PREDICTION METHOD, AND DEVICE FOR PERFORMING SAME

(71) Applicant: KT CORPORATION, Gyeonggi-do (KR)

(72) Inventor: Yong-Lae CHO, Gyeonggi-do (KR)

(73) Assignee: KT CORPORATION, Gyeonggi-do (KR)

(21) Appl. No.: 15/746,524

(22) PCT Filed: Jul. 11, 2016
(86) PCT No.: PCT/KR2016/007501

§ 371 Date: Jan. 22, 2018

(30) Foreign Application Priority Data
Jul. 22, 2015 (KR) 10-2015-0103951

(51) Int. Cl. G06F 19/18; G16H 50/30

(52) U.S. Cl. CPC G06F 19/18; G01N 2800/52; G01N 2800/50; G16H 50/30

(57) ABSTRACT

A method for predicting disease risk and an apparatus for performing the same are disclosed. The method for predicting disease risk is a method for predicting disease risk using an apparatus for computer-based disease risk analysis connected to a network, which includes, selecting at least one disease-related disease-variation, predicting disease risk using the at least one disease-variation, providing the predicted results of disease risk to a user terminal through the network, receiving feedback from the user terminal whether a user has developed a disease, and identifying the developed disease through the feedback and setting a weight value on at least one disease-variation used in predicting the risk of the actually-developed disease.

### TECHNICAL FIELD

The present disclosure relates to a method for predicting disease risks and an apparatus for performing the same, and more specifically, to a method for predicting dielectric material-based disease risks.

### BACKGROUND ART

Due to the development of genome sequencing technology, many personal genome services (PGS), which predicts diseases based on individual dielectric material information, have developed.

Generally, the probability of disease development is calculated as an “average population disease risk”×“relative risk”. However, these techniques are now being questioned with respect to their accuracy. The results of disease prediction differ from company to company even for the same patient. This is because the results may vary depending on how the disease-related genetic variations are selected.

With respect to disease risk analysis based on genetic information, if a disease is caused by a single gene abnormality, the result is explicit, whereas if a disease is caused by a complex gene abnormality, the test result may differ for each PGS company. For example, the selection of variations (variables) in the list of genetic variations reported to be related to type 2 diabetes significantly affects disease risk analysis.

TABLE 1

| Gene | Variation | Risk | Company A | Company B | Company C |
|---|---|---|---|---|---|
| TCF7L2 | rs79031 | 34% | ◯ | | |
| SLC30A8 | rs13266 | 37% | | | ◯ |
| EPO | rs16176 | 57% | ◯ | ◯ | ◯ |
| FTO | rs99396 | 58% | | ◯ | ◯ |
| | | Total | 45% | 57.5% | 50.6% |

Table 1 is a list of genetic variations known to be related to type 2 diabetes, and companies may predict various results based on the selection of the disease-variation list. It is also very important to appropriately select the variations that affect each race, as different diseases may develop for different races.

As such, it has become a significant problem in disease prediction services, because the results may differ for each company depending on which variation was selected for each disease.

Additionally, in the process of selecting the disease-variation, the accuracy of disease prediction cannot be increased simply by information known to be a risk in a public database (DB) in the genomic field and other various disease DB.

### DISCLOSURE

### Technical Problem

Accordingly, a feature of the present disclosure is to provide a method for predicting disease risks capable of increasing result accuracy with respect to genetic variations used for the prediction of diseases based on genetic information by assigning weight values from feedback results from a user, and an apparatus for performing the same.

### Technical Solution

According to a feature of the present disclosure, a method may be provided for predicting disease risk using an apparatus for computer-based disease risk analysis connected to a network. The method may include selecting disease-related disease-variations; predicting a disease risk using the disease-variations; providing the predicted disease risk results to a user terminal through the network; receiving a feedback from the user terminal on whether a user has developed a disease; and identifying the developed disease through the feedback, and setting a weight value on at least one disease-variation used in predicting the risk of the developed disease, wherein the selecting comprises selecting one having a relatively high weight from the disease-variations.

The providing and the receiving feedback may be implemented through mobile services.

The selecting may include: at a first selection, examining disease-related genes and variations; assigning a medical ground level and a basic weight value to each examined disease-variation; selecting the disease-variations to be used in predicting disease risk based on a medical ground level; and generating a product based on the selected disease-variations, and the predicting may include predicting a risk using the generated product.

The examining the disease-related genes and variations may include examining from a plurality of websites and databases where information on disease-related genes and mutations are stored, examining research articles with respect to a correlation between diseases and race, and collecting review information by experts; and the medical ground level is assigned based on number of samples, animal experiment certificates, a statistical significance, number of articles reported in journals, whether the articles have been reported to academic conferences with a high impact factor, and the medical ground level reported to other database based on the collected information.

The generating a product may include generating the product to include different combinations of disease-variations related to a disease, where each combination is matched with the product identification information including a product unique ID and a product version information and includes the medical ground level, and the weight value, number of discovery of variations, number of times of providing the product, the development of a disease, and a final relevance score. Herein, the final relevance score is information used to select the disease-variations to be used in predicting the disease risk.

The final relevance score may be calculated using a correlation coefficient of the medical ground level, a correlation coefficient of the weight value, the medical ground level, and the weight value.

The receiving feedback may include: receiving information for identifying the product related to the disease developed to the user, a disease name, a disease-variation ID, and whether the disease has developed, and

The selecting may include: if the selection is not the first selection, increasing a weight value to disease-variations related to the developed disease confirmed through the user feedback information, and reselecting the disease-variation to be used in predicting disease risk based on the weight value.

The weight value may be calculated based on whether the disease has developed and a number of variations discovered.

The predicting disease risk may include generating a user variation ID list by matching the genes and disease-variations related to the first-selected or reselected disease with the user gene information; if the disease is a complex disease and the disease-variations comprised in the user variation ID list are not comprised in the product, determining the variations as not related to the disease and excluding the variations accordingly; if the disease is a complex disease and the disease-variations comprised in the user variation ID list are comprised in the product, determining the variations as related to the disease and predicting disease risk based on the disease-variations comprised in the product; if the disease is a rare disease and the disease-variations comprised in the user variation ID list are comprised in the product, classifying disease risk as high risk; if the disease is a rare disease and the disease-variations comprised in the user variation ID list are not comprised in the product, but the disease-variations affect protein structures or cause loss of functions, classifying the subject disease as a high risk group; and if the disease is a rare disease and the disease-variations comprised in the user variation ID list are not comprised in the product, or the disease-variations do not affect protein structures or cause loss of functions, determining the variations as not related to the disease and excluding the variations accordingly.

The providing may include providing a result report comprising a product version ID, a disease name, a variant ID, and a disease risk as mobile services through a smart phone application.

According to another feature of the present disclosure, an apparatus may be provided for computer-based disease risk analysis connected to a network. The apparatus may include a disease-variation selecting data-base (DB) configured to store a disease-variation table, wherein the disease-variation table is a reference information table to set medical ground levels and disease-variation information to use in predicting disease risk; a disease-variation selecting unit configured to select disease-variations related to diseases using the reference information table and includes the selected disease-variations information to the disease-variation table; a disease risk predicting unit configured to predict a disease risk using the disease-variations in the disease-variation table; a providing unit configured to provide results of disease risk predicted by the disease risk predicting unit to a user terminal through the network; a user feedback unit configured to receive a feedback as to whether a disease has developed in a user from the user terminal; and a weight value setting unit configured to confirm whether a disease has developed through the feedback and sets a weight value to at least one disease-variation used in predicting the risk of the developed disease. The disease-variation selecting unit may be configured to select one having a relatively high weight value among the disease-variations comprised in the disease-variation table.

The reference information table may include medical ground levels, which are a criteria representing an extent of strength with respect to a disease-variation correlation set based on ground levels reported in other disease-related DB, which represents cases where information in other disease DB that include information on number of samples used in disease-variation correlation studies, animal experiment certificates that represent cases where studies on genetic functions are performed through animal experiments, and statistical significance of disease-variation correlation studies and disease-variation correlation. The disease-variation selecting unit may examine disease-related genes and variations from a plurality of websites and databases in which information on disease-related genes and mutations is stored, further examines research articles with respect to a correlation between diseases and race, and collects review information by experts, and selects disease-variations related to diseases based on the collected information and the medical ground levels.

The disease-variation table may store an ID and a version information of a product consisting of a combination of mutually-different diseases-variations, disease names, ID of disease-variations related to diseases, a medical ground level for each of the disease-variations, a weight value for each of the disease-variations, number of cases where the subject disease-variation are actually found among people who have used the product, a number that the product is offered, and a final relevance score calculated using the number of people in whom the disease was actually developed and the weight value. The disease-variation selecting unit may select disease-variations in an order from a highest relevance score to a lowest relevance score.

The user feedback unit may receive user feedback information, wherein the ID and version information of the product related to the disease developed in a user, a name of the disease, IDs of the disease-variations, and whether the disease has developed. The weight value setting unit may increase weight values for the disease-variations related to the developed disease as confirmed through a user feedback information.

The weight value setting unit may set the weight values that are calculated on whether the disease has developed and number of variations discovered to the disease-variations.

### Advantageous Effects

According to an embodiment of the present disclosure, unlike a typical method which receives feedback on the user's satisfaction with respect to disease risk prediction, in the present disclosure, whether a disease has actually developed is received as feedback, and thus, by assigning weight values to disease-variations related to disease development used at the early stage, and is preferentially used in predicting disease risk of disease-variation with a high weight value. Then, by utilizing weight values in selecting genetic variations used in predicting disease risk based on genetic information, the accuracy of predicting disease risk is improved as the accumulated amount of the results of disease development increases.

### DESCRIPTION OF THE DRAWINGS

FIG. 1 is a block diagram depicting a feature of an apparatus for disease risk analysis according to an exemplary embodiment of the present disclosure.

FIG. 2 is a flowchart showing a method for predicting disease risk according to an exemplary embodiment of the present disclosure.

FIG. 3 is a flowchart showing a process of selecting disease-variations according to an exemplary embodiment of the present disclosure.

FIG. 4 is a flowchart detailing step S203 of FIG. 3.

FIG. 5 is a reference information table showing components for selecting disease-variation according to an exemplary embodiment of the present disclosure.

FIG. 6 is a disease-variation table showing components according to an exemplary embodiment of the present disclosure.

FIG. 7 is a flowchart showing the process of predicting disease risk according to an exemplary embodiment of the present disclosure.

FIG. 8 is an illustration of predicted results of disease risk according to an exemplary embodiment of the present disclosure to a user.

FIG. 9 is an illustration of user feedback according to an exemplary embodiment of the present disclosure.

FIG. 10 is a flowchart showing a process of user feedback according to an exemplary embodiment of the present disclosure.

FIG. 11 shows data format for user feedback.

FIG. 12 is an updated illustrative flowchart of a disease-variation table according to an exemplary embodiment of the present disclosure.

FIG. 13 is a block diagram showing a feature of an apparatus for disease risk analysis according to another exemplary embodiment of the present disclosure.

### MODE FOR INVENTION

In the following detailed description, only certain exemplary embodiments of the present disclosure are shown and described by way of illustration. However, the present disclosure may be implemented in many different forms and is not limited to the exemplary embodiments described herein. Accordingly, the drawings and description are to be regarded as illustrative in nature and not restrictive. Like reference numerals designate like elements throughout the specification.

In the entire specification, In addition, unless explicitly described to the contrary, the word “comprise” and variations such as “comprises” or “comprising”, will be understood to imply the inclusion of stated elements but not the exclusion of any other elements.

Additionally, the terms of “ . . . unit” and “ . . . module” described herein refer to a unit for handling at least one function or action, and these can be implemented by hardware or software or a combination of hardware and software.

Hereinafter, the apparatus for analyzing disease risk according to an exemplary embodiment of the present disclosure and a method thereof in detail.

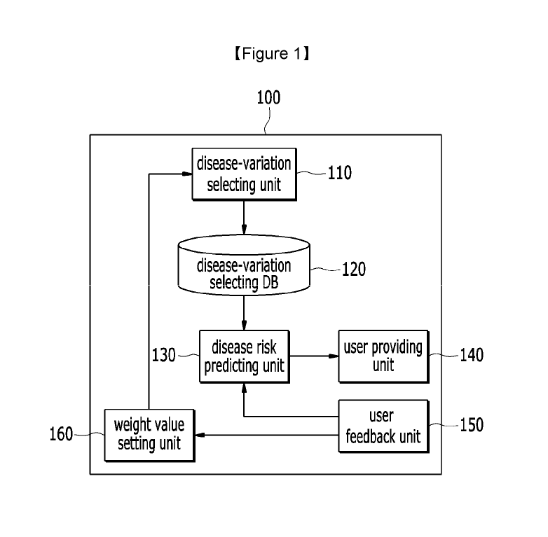

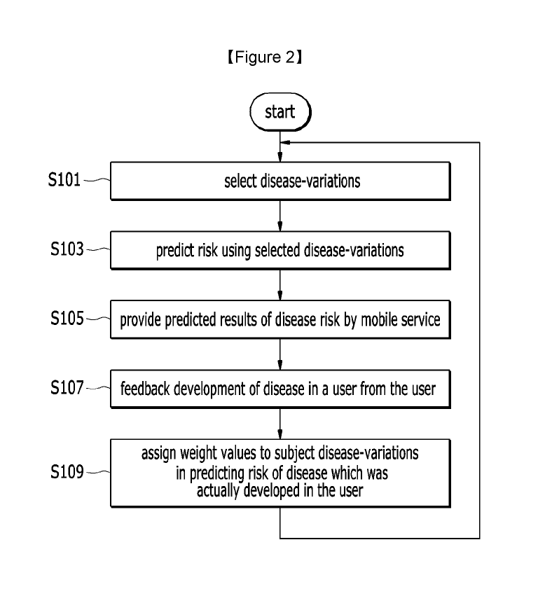

FIG. 1 is a block diagram showing a feature of an apparatus for disease risk analysis according to an exemplary embodiment of the present disclosure, and FIG. 2 is a flowchart showing a method for predicting disease risk according to an exemplary embodiment of the present disclosure.

Referring to FIG. 1, an apparatus for analyzing disease-variation 100 includes a disease-variation selecting unit 110, a disease-variation selecting database (DB) 120, a predicting disease risk unit 130, a user providing unit 140, a user feedback unit 150, and a setting weight values unit 160.

Referring to FIG. 2, the unit disease-variation selecting unit 110 selects disease-variations related to diseases in step S101. The selected disease-variations are stored in the disease-variation selecting DB 120. Hereinafter, variations related to diseases are collectively described as “disease-variation”.

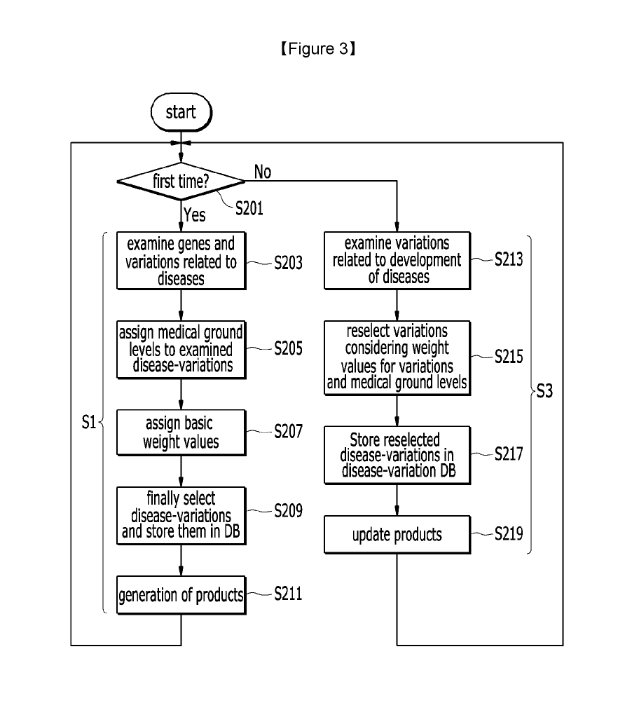

The disease-variation selecting unit 110 selects disease-variation related to a disease(s) by considering various medical related data, weight values for variations, etc. The selection process will be described in detail with reference to FIG. 3 below.

In step S103, the disease risk predicting unit 130 predicts disease risk based on the disease-variations selected in step S101. This process predicts mutually different disease risks according to disease characteristics using the selected disease-variations.

The user providing unit 140 provides the predicted results of disease risk predicted in step S103 to a user terminal (not shown), e.g., a mobile service (S105). For example, the mobile service may be implemented in the form of a mobile web or smart phone application in a mobile terminal.

In step S107, the user feedback unit 150 receives feedback with respect to the development of a disease from the user terminal (not shown).

The weight values setting unit 160 in step S109 allocates weight values to the disease-variations used in predicting disease risk which was provided to a user, in which a disease was actually developed, and received a feedback in step S107. Then, the disease-variations for which weight values are allocated are selected to predict disease-variations and are used for the prediction of disease risk.

For example, assuming if variations such as A, B, C, D, E, and F are present as causative genetic variations of diabetes, the causative genetic variations of diabetes for patient no. 1 may be A, C, and ID, those for patient no. 2 may be B, E, and F; and those for patient no. 3 may be A, D, and F.

As such, there are diverse causative variations for diabetes in patients, and thus it is difficult to know which variation it may be, and the difference in variation combination pattern may vary among different races.

For example, the apparatus for analyzing disease-variation 100 according to an exemplary embodiment of the present disclosure selects variations A, B, D, and F as causative variations for diabetes with respect to Koreans at the initial stage, and allocates weight values to A, B, D, and F, which were selected at the time of actual development of diseases through user feedback, while performing disease risk prediction services, and thereby preferentially use them in predicting diseases in Koreans, rather than other variations such as C and E. This increases the accuracy of the predicted results of disease risk, which varies among different races and individuals.

FIG. 3 is a flowchart showing the process of selecting disease-variations according to an exemplary embodiment of the present disclosure, which represents the action of the disease-variation selecting unit 110 of FIG. 1, and specifically represents step S101 of FIG. 2.

Referring to FIG. 3, the process of selecting disease-variations is largely classified into two (2) processes, which includes a process for selecting novel variations related to diseases (S1) and a process for reselecting variations among the variations related to diseases considering weight values, medical ground levels, etc. (S3).

First, the apparatus for analyzing disease-variation 110 determines whether the selection of disease-variation is a first-time selection (S201). That is, it is a step to determine whether the step corresponds to S1 or S2.

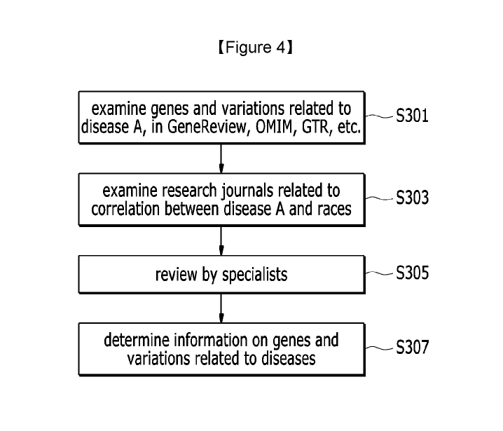

After it is determined to be the first step S203 (S1) for selecting disease-variation, the disease-variation selecting unit 110 examines genes and variations related to diseases by various conditions. In particular, step S203 will be described later referring to FIG. 4.

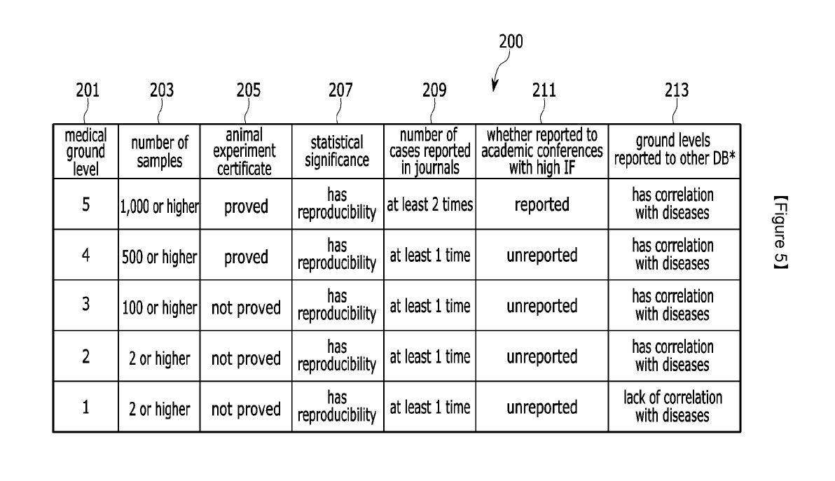

The disease-variation selecting unit 110 allocates medical ground levels to disease-variations examined in step S203 (S205). In particular, medical ground levels may be allocated to disease-variations based on the reference information table 200 of FIG. 5; for example, if the number of examined samples of disease-variation is 500 or higher, have approved animal experiments, have statistical significance, the number of cases reported in journals is three (3), and is reported to academic conferences with high IF, and have a correlation with diseases, then the corresponding conditions are compared with the reference information table 200, and the medical ground level is allocated as four (4).

Then, the disease-variation selecting unit 110 allocates a basic weight value (e.g., 1) to disease-variations to which medical ground levels are assigned (S207).

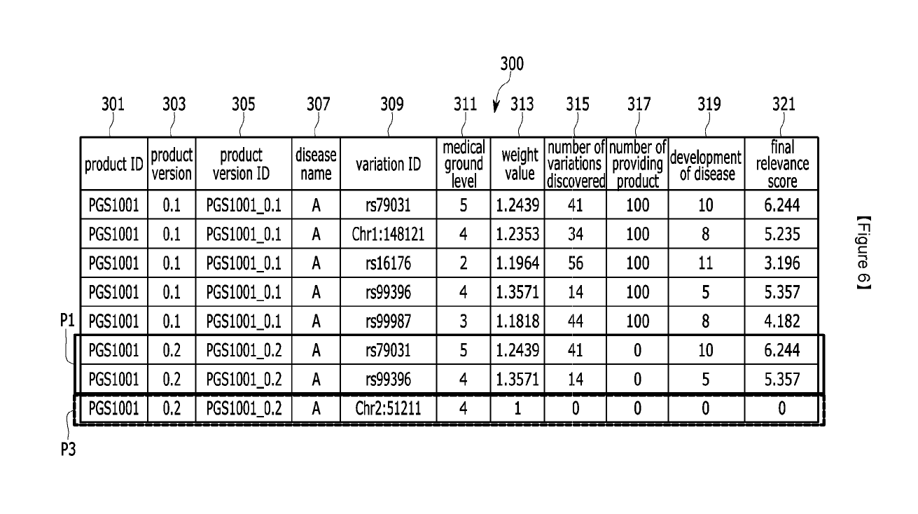

Then, the disease-variation selecting unit 110 stores the finally-selected disease-variations in the disease-variation selection DB 120 and generates products (S211). As such, the generated products are generated in a disease-variation Table 300, as shown in FIG. 6.

In particular, the disease-variation selection DB 120 stores the reference information Table 200 and the disease-variation Table 300 of FIG. 6.

Meanwhile, the disease-variation selecting unit 110 examines variations related to development of diseases in step S201, if it is not the first time, that is, if it is determined that it is a process for reselecting disease-variation, S213 (S3). That is, in step S107 of FIG. 2, the disease-variations used in predicting the disease risk of the actually-developed disease through user feedback are confirmed.

The disease-variation selecting unit 110 reselects the disease-variations to be used in predicting disease risk among the disease-variations with high weight values examined in step S213 considering medical ground levels (S215).

The disease-variation selecting unit 110 stores the disease-variations reselected in step S215 in the disease-variation selection DB 120 (S217) and updates products (S219). As such, the updated products are renewed in the disease-variation Table 300 as shown in FIG. 6.

FIG. 4 is a flowchart detailing the step of S203 of FIG. 3.

Referring to FIG. 4, the disease-variation selecting unit 110 examines information on genes and variations related to diseases, e.g., websites and database in which information on disease-related genes and mutations may be stored (S301).

In particular, the unit for selecting disease-variation 110 may include GeneReview site (http://www.ncbi.nlm.nih.gov/books/) in which the correlation between diseases and genes are reviewed by experts, OMIM (http://www.ncbi.nlm.nih.gov/omim), Pubmed Site (http://pubmed.com) in which information on rare diseases that comply with Mendelian principles is collected, GTR (Genetic Testing Registry) (http://www.ncbi.nlm.nih.gov/gtr/) in which information on test items being performed by gene examination organizations in the world.

Then, the disease-variation selecting unit 110 examines research articles on correlation between diseases and races (S303). The disease-variation selecting unit 110 determines the disease-variation through expert review S305, etc. based on information collected through steps S301 and S303 (S307).

In particular, for steps S301, S303, and S305, various information examined by a user through an apparatus, for example, a computer, tablet, mobile device, etc., in which an input device such as a keyboard, etc. and programs for inputting, storing, and outputting through the input device are installed, and monitors, may be used. Alternatively, various information disclosed in the network through programs may be collected and subjected to proofreading by experts.

FIG. 5 is a reference information table showing components for selecting disease-variation according to an exemplary embodiment of the present disclosure.

Referring to FIG. 5, the disease-variation selecting unit 110 assigns medical ground levels to disease-variations collected in FIG. 3 and FIG. 4 based on the reference information stored in the reference information Table 200.

In particular, the reference information Table 200 consists of a plurality of items, and these plurality of items include: a medical ground level 201, a sample number 203, an animal experiment certificate 205, statistical significance 207, a number of cases reported in journals 209, whether it has been reported to academic conferences with high impact factor (IF), and a ground level that has been reported to other disease-related DB 213.

Medical ground level 201 is a measure of strength, which represents the level of disease-variation correlation. Medical ground level 201 is not information representing the stage of disease risk. Medical ground level 201 is used as a reference material when disease-related variations are finally selected.

The sample number 203 refers to the number of samples used in disease-variation correlation studies. For example, if the people infected with disease A are 100 and those not infected with disease A are 150, the sample number is recorded as 250.

Animal experiment certifications 205 represent the case where genetic functions are studied through animal experiments, etc. in disease-variation correlation studies.

Statistical significance 207 represents whether there was a statistical difference in disease-variation correlation studies. For example, Genome-wide Association Study (GWAS) study reveals whether there was a significant difference in P-value or whether there was a significant difference in linkage analysis.

The ground level reported in a disease DB 213 represents a case where information in other DBs containing disease-variation correlation disease-variation correlation is present. For example, it is expressed as having correlation or having no correlation depending on the presence of correlation in ClinVar DB.

FIG. 6 is a disease-variation table showing the components according to an exemplary embodiment of the present disclosure.

Referring to FIG. 6, the disease-variation Table 300 stores the disease-variation information selected in step S101 of FIG. 2 and steps S209, S211, and S217 of FIG. 3, for utilization in predicting disease risk.

The disease-variation Table 300 consists of a plurality of items, and these plurality of items include a product ID 301, a product version 303, a product version ID 305, a disease name 307, a variation ID 309, a medical ground level 311, a weight value 313, a number of variations discovered 315, a number of provision of products 317, a presence of development of diseases 319, and a final relevance score 321.

The product ID 301 stores unique IDs for the products. The product ID 301 may be classified into subjects, type of diseases, etc. and consists of a combination of disease-variations.

The product version 303 stores information on product versions.

The product version ID 305 stores unique IDs that represent product versions. In particular, the unique IDs are assigned per product version by combining product IDs and product versions.

For the disease names 307, disease information, which is a subject for predicting disease risk, is recorded. For example, a disease name such as type 1 diabetes or a disease code that represents type 1 diabetes is recorded.

For the variation ID 309, a unique ID of the variation related to the disease recorded in the disease name 307 is recorded. In particular, the variation refers to a sequence in which the genome sequence of an individual is different compared to that of the human genome reference, and it means a sequence related to characteristics, diseases, etc. of the individual.

The variation ID is expressed in two types, and the first type may be expressed as a chromosomal number (position of variation within the chromosome), and the second type may be expressed as an rslD, i.e., the ID of the single nucleotide polymorphism database (dbSNP) DB. In particular, the dbSNP is a variation DB provided by the U.S. National Center of Biotechnology Information. Single nucleotide polymorphism (SNP) shares its lists through the dbSNP (http://www.ncbi.nlm.nih.gov/projects/SNP/snp_summary.cgi).

The medical ground level 311 records medical ground level information with respect to correlation between the disease name 307 and the variation recorded in the variation ID 309, and it is set based on the information recorded in medical ground level 201 of FIG. 5. For example, when variation ID “rs79031” is based on the reference information Table 200, if the sample number is 1,000 or higher, has approved animal experiment(s), has statistical significance, the number of cases reported to journals is two (2) or higher, and it has been reported to academic conferences with a high IF, and has disease correlation according to the ground level reported to a disease DB, the medical ground level is set as “5”.

The weight value 313 represents weight value information with respect to disease-variation correlation.

The number of variations discovered 315 represents the number of cases where the subject variation has been actually discovered among the people who have used the subject product version.

The number of provision of products 317 represents the number of people who have used the subject product version.

The presence of development of diseases 319 represents the number of people in whom the disease has actually developed.

The final relevance score 321 represents the final relevance score. The disease-variation to be used in predicting disease risk is determined based on the final relevance score.

The disease-variation selecting unit 110 calculates the final relevance score considering the medical ground levels and weight values.

Final Relevance Score=αX+βY [Equation 1]

In the above equation, α represents a correlation coefficient of the medical ground level, β represents a correlation coefficient of the weight value, X represents a value of the medical ground level, and Y represents a value of the weight value.

In particular, the correlation coefficient of the medical ground level and the correlation coefficient of the weight value refer to correlation coefficients, which are constant values obtained by logistic regression statistical analysis between the medical ground level and the weight value.

X is a value recorded in item 311 of the disease-variation Table 200 and Y is a value recorded in item 313 of the disease-variation Table 200.

For example, when a has a correlation coefficient of 1 and β has a correlation coefficient of 2, according to the disease-variation Table 200, the medical ground level is 5 and the weight value is 1.2439, with respect to the variation rs79031. Accordingly, the final relevance score for the variation rs79031 is calculated as 1×5+2×1.2439=7.488.

The disease-variation selecting unit 110 is referred to the disease-variation selection process for next product versions (0.1, 0.2) 303 in the order from the highest final relevance score 321 to the lowest final relevance score 321, considering the medical ground level, the weight value, etc. of the disease-variation Table 200.

For example, “rs79031” and “rs99396”, which have high final relevance score among the 5 variations used in the PGS1001 product 0.1 version, are preferentially included at the time of product 0.2. Accordingly, in the disease-variation Table 200, the 0.2 version includes the existing variation P1, which was used in the 0.1 version, and a novel variation P3, which was used only in the 0.2 version.

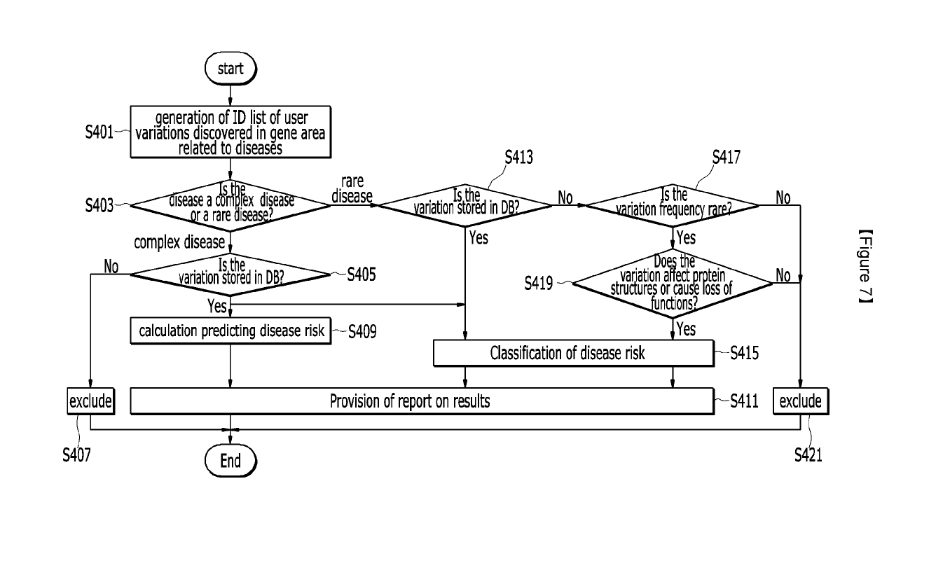

FIG. 7 is a flowchart showing the process of predicting disease risk according to an exemplary embodiment of the present disclosure, which represents the action of the disease risk predicting unit 130 and step S103 of FIG. 2 in detail.

Referring to FIG. 7, the disease risk predicting unit 130 generates a user variation ID list in which is user variation IDs discovered in gene regions related to diseases (S401). The disease risk predicting unit 130 generates a user variation ID list by matching the genes related to diseases and disease-variations selected by the disease-variation selecting unit 110 with the user gene information. The user variation ID consists of chromosomal position or rslD, as explained above.

The disease risk predicting unit 130 determines whether the subject disease to be predicted is a rare disease or complex disease (S403).

If the disease is determined to be a complex disease, that is, a disease that occurs due to a complex cause such as genetic, environmental factors, etc., it is determined whether the user variation included in the user variation ID list is a variation stored in the disease-variation Table 300 (S405). In particular, if the variation is not a stored variation, the variation is determined to be a normal variation not relating to a disease and thereby the subject variation is excluded from risk prediction (S407).

In contrast, if the variation is a stored variation, the prediction of disease risk is calculated using the ID matched in the disease-variation Table 300 (S409), and the results report including the calculated results are provided to the user (S411).

In particular, a post-test probability method, a calculation using a ration, a calculation method using relative risk, etc. may be used for the calculation of disease risk prediction, but the method is not limited thereto and various other methods for predicting disease risk may be used.

If the disease is determined to be a rare disease in step S403, the disease risk predicting unit 130 determines whether the user variation included in the user variation ID list is a variation stored in the disease-variation Table 300 (S413). If the variation is a stored variation, the user variation ID list is a factor that causes a disease and thus the disease risk predicting unit 130 classifies the disease into a high risk group (S415). And the results report including the classification results are provided to the user (S411).

If the variation is not a variation stored in the unit for predicting disease risk 130 in step S413, the variation may be a variation which may not have been previously known, that is, a variation which is specifically discovered in a certain individual, and the unit for predicting disease risk 130 determines whether the variation frequency is rare (S417).

In particular, the variation frequency is confirmed using 1000 Genome DB(http://www.1000genomes.org/), ExAC DB(http://exac.broadinstitute.org/), etc. When the variation frequency is 0.05 or below 0.01 or less, the variation is defined to be rare considering the occurrence rate of rare diseases.

Once the variation frequency is determined to be rare, the disease risk predicting unit 130 determines whether the user variation ID list has an effect on protein structures (protein altering) or causes the loss of functions (S419).

If the user variation ID list has an effect on protein structures or causes the loss of functions in step S419, the subject disease is classified as a high risk group (S415), and the results report is provided to the user (S411).

In contrast, if the variation frequency is not rare in step S417 or if the variation frequency does not have an effect on protein structures or cause the loss of functions in step S419, the subject variation is excluded (S421).

Meanwhile, steps S401 and S421 may be performed with respect to diseases included in products in the disease-variation Table 300.

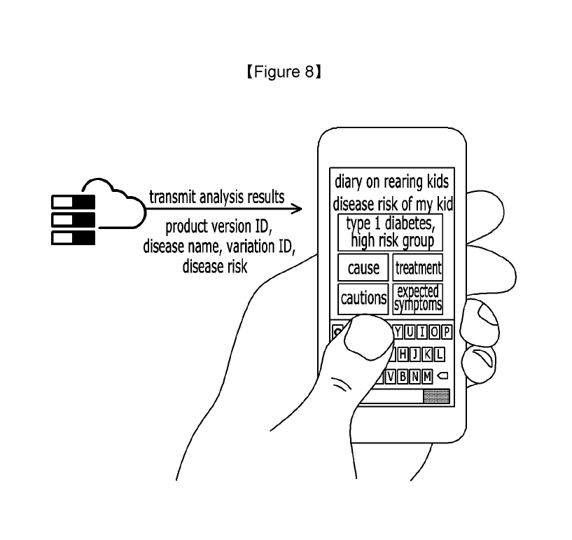

When the disease is a rare disease, if the subject user variation ID is included in the disease-variation Table 300, or the variation frequency is rare, and the subject variation has an effect on protein structures or causes the loss of functions, the disease risk predicting unit 130 classifies the disease as a high risk group. And when the disease is a complex disease, the disease is classified as relative risk, whereas when the disease is a rare disease, the disease is classified as high risk group/low risk group, etc. and the results report including the subject classification results are provided (S411). The results report may be implemented as shown in FIG. 8.

FIG. 8 is an illustrative flowchart providing the predicted results of disease risk according to an exemplary embodiment of the present disclosure to a user, which represents the action of the a user providing unit 140 of FIG. 1 and represents step S105 of FIG. 2.

Referring to FIG. 8, the user providing unit 140 receives the analysis results from the disease risk predicting unit 130 and then provides the results to a user terminal (not shown). In particular, the user providing unit 140 can provide the results report through an application which is installed in the user terminal (not shown), e.g., an application for a diary for rearing kids. In particular, the user providing unit 140 can provide a results report including product version ID, disease name, variation ID, and disease risk.

The user providing unit 140 collects the presence of development of diseases in the future while providing mobile care services with respect to the subject disease in applications such as mothers' diary, diary for rearing kids, etc., according to the predicted results of disease risk. In an exemplary embodiment, if the analysis results in the analysis service reveals to be “type 1 diabetes of a high risk group”, the user providing unit 140 transmits the subject information by mobile communication. Various care service information such as “causes”, “treatment”, “cautions”, “expected symptoms”, etc., with respect to “type 1 diabetes” are provided.

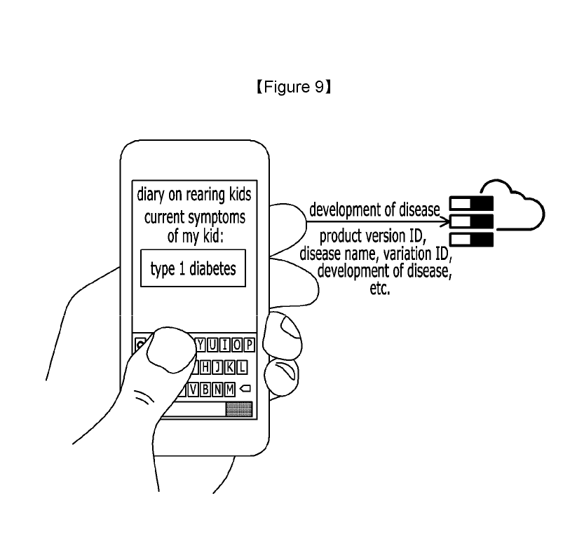

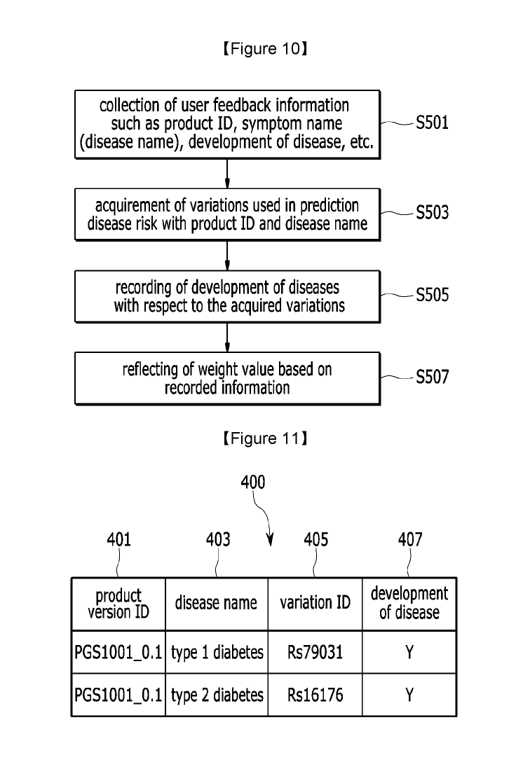

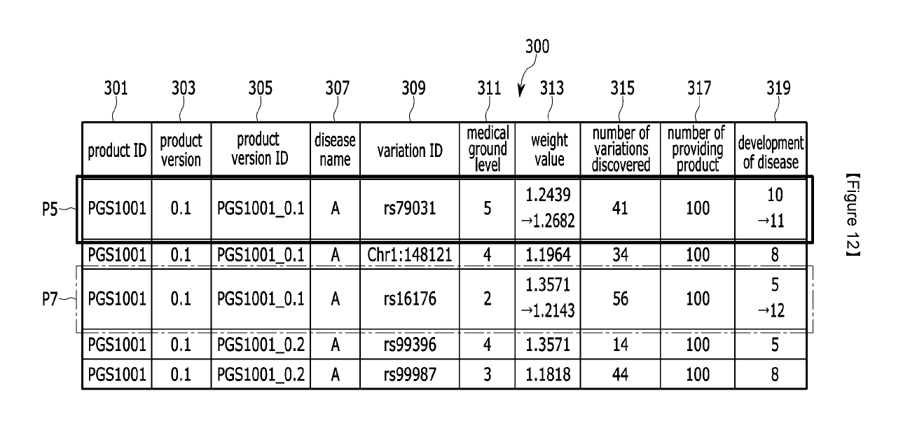

FIG. 9 is an illustrative flowchart showing the user feedback according to an exemplary embodiment of the present disclosure; FIG. 10 is a flowchart showing the process of user feedback according to an exemplary embodiment of the present disclosure; FIG. 11 shows a data format for user feedback; and FIG. 12 is an updated illustrative flowchart of a disease-variation table according to an exemplary embodiment of the present disclosure.

FIG. 9 and FIG. 10 represent the action of the user feedback unit and represents step S107 of FIG. 2 in detail.

Referring to FIG. 9, the user transmits the presence of the development of a disease to the user feedback unit 150 through the user terminal (not shown). In particular, the presence of the development of a disease includes the product version ID, the disease name, the variation ID, and the presence of the development of a disease. The user checks whether a disease has actually developed while receiving the mobile care service. The presence of the development of a disease may be determined by directly selecting the disease in the user terminal (not shown), or by assuming the presence of the development of a disease through related questionnaire, etc. Once the presence of the actual development of a disease is determined, the product version ID, the disease name, the variation ID, and the presence of the development of a disease, etc. are transmitted to the user feedback unit 150.

Referring to FIG. 10, the user feedback unit 150 collects user feedback information such as the product ID, the symptom name (disease name), the presence of the development of a disease, etc. related to the actually-developed disease from the user terminal (not shown) (S501).

In particular, the information being collected may be in the same format as shown in FIG. 11.

Referring to FIG. 11, user feedback information 400 includes the product ID 401, the disease name 403, the variation ID 405, and the presence of the development of a disease 407. In particular, the product ID 401, the disease name 403, the variation ID 405, and the presence of the development of the disease 407 include the product information 401, the information on the developed disease 403, and the information on the variation used for predicting the risk of the disease developed 405, which are related to the actually-developed disease, in the predicted report of disease risk provided to the user.

Again, referring to FIG. 10, the user feedback unit 150 records the presence of the development of a disease with respect to disease-variation obtained (S503) based on the user feedback information collected in step S501 to the item 319 in the presence of the development of a disease of the disease-variation Table 300, which corresponds to the product version ID 401, the disease name 403, and the variation ID 405 (S505). And the weight values setting unit 160 calculates the weight value based on the recorded information and reflects on the weight value item 313 of the disease-variation Table 300 (S507). That is, the weight values setting unit 160 assigns a weight value with respect to a subject disease-variation in the disease-variation Table 300, based on the information received from the user. The value of the presence of the development of a disease 319 with respect to the item, in which the information on the product version ID 401, the disease name 403, and the variation ID 405 received from the user in the disease-variation Table 300 are matched, is increased. The presence of the development of a disease 319 increases as many as the number of users in whom the user feedback information was received. And the calculated weight values are updated in the weight value 313.

In particular, the weight value is calculated by Equation 2 below.

Weight Value=1+(presence of development of a disease/the number of variations discovered) [Equation 2]

In particular, the presence of development of a disease represents the number of people in which the disease recorded in the presence of development of a disease 319 in the disease-variation Table 300 has actually developed. And the number of variations discovered represents the number of the subject variation that has been actually discovered among the people who have used the product version recorded in the number of variations discovered 315 of the disease-variation Table 300.

Referring to FIG. 12, if Equation 2 is applied to “variation ID=rs79031” and “variation ID=rs16176”, respectively, their weight values are updated to 1.2682 and 1.2143, respectively.

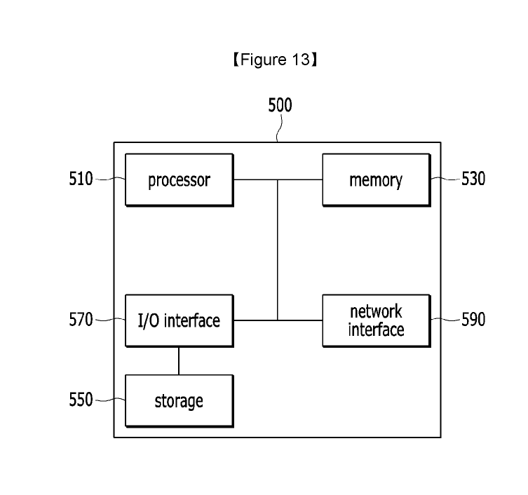

Meanwhile, FIG. 13 is a block diagram depicting the feature of an apparatus for disease risk analysis according to another exemplary embodiment of the present disclosure.

Referring to FIG. 13, an apparatus for analyzing disease risk 500 includes a processor 510, a memory 530, at least one storing device 550, an input/output (I/O) interface 570, and a network interface 590.

The processor 510 may be implemented in the form of a central processing unit (CPU) or other chip sets, microprocessor, etc. and may be implemented with RAM, such as the dynamic random access memory (DRAM), rambus DRAM (RDRAM), synchronous dynamic DRAM (SDRAM), static RAM (SRAM), etc.

The storage device 550 may be implemented in the form of a permanent or volatile storage device including optical disks such as hard disks, compact disk read only memory (CD-ROM), CD rewritable (CD-RW), digital video disk ROM DVD-ROM( ) DVD-RAM, DVD-RW disk, blue-ray disks, flash memory, and various forms of RAM.

Additionally, the I/O interface 570 allows for the processor 510 and/or memory 530 to be accessed to the storage device 550, and the network interface 590 allows for the processor 510 and/or memory 530 to be accessed to the network (not shown).

In this case, the processor 510 loads the program commands for implementing at least part of the functions of the unit for selecting disease-variation 110, the unit for predicting disease risk 130, the unit for providing to a user 140, the unit for user feedback 150, and the unit for setting weight values 160, to the memory 530, and locating the function of the disease-variation selection DB 120 at the storage device 550, thereby controlling the explained action to be performed referring to FIG. 1.

Additionally, the memory 530 or storage device 550 may be linked with the processor 510 thereby allowing the functions of the disease-variation selecting unit 110, the disease risk predicting unit 130, the user providing unit 140, user feedback unit 150, and the weight values setting unit 160 to be performed.

The processor 510, memory 530, at least one storage device 550, input/output (I/O) interface 570, and network interface 590 illustrated in FIG. 13 may be implemented in single apparatus or implemented after being dispersed into a plurality of apparatus.

The embodiments of the present disclosure described above are not only implemented by an apparatus and a method, but they may be implemented through any program which can implement the functions corresponding to the features of the exemplary embodiments of the present disclosure or recording medium on which the programs are recorded.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1. A method for predicting disease risk using an apparatus for computer-based disease risk analysis connected to a network, comprising:
selecting disease-related disease-variations;
predicting a disease risk using the disease-variations;
providing the predicted disease risk results to a user terminal through the network;
receiving a feedback from the user terminal on whether a user has developed a disease; and
identifying the developed disease through the feedback, and setting a weight value on at least one disease-variation used in predicting the risk of the developed disease,
wherein the selecting comprises selecting one having a relatively high weight from the disease-variations.

2. The method of claim 1, wherein the providing and the receiving feedback are implemented through mobile services.

3. The method of claim 1,
wherein the selecting comprises:
at a first selection, examining disease-related genes and variations;
assigning a medical ground level and a basic weight value to each examined disease-variation;
selecting the disease-variations to be used in predicting disease risk based on a medical ground level; and
generating a product based on the selected disease-variations, and
wherein the predicting comprises:
predicting a risk using the generated product.

4. The method of claim 3, wherein the examining the disease-related genes and variations includes examining from a plurality of websites and databases where information on disease-related genes and mutations are stored, examining research articles with respect to a correlation between diseases and race, and collecting review information by experts; and
the medical ground level is assigned based on number of samples, animal experiment certificates, a statistical significance, number of articles reported in journals, whether the articles have been reported to academic conferences with a high impact factor, and the medical ground level reported to other database based on the collected information.

5. The method of claim 4,
wherein the generating a product comprises generating the product to include different combinations of disease-variations related to a disease, where each combination is matched with the product identification information including a product unique ID and a product version information and includes the medical ground level, and the weight value, number of discovery of variations, number of times of providing the product, the development of a disease, and a final relevance score, and
wherein the final relevance score is information used to select the disease-variations to be used in predicting the disease risk.

6. The method of claim 5, wherein the final relevance score is calculated using a correlation coefficient of the medical ground level, a correlation coefficient of the weight value, the medical ground level, and the weight value.

7. The method of claim 5,
wherein the receiving feedback comprises: receiving information for identifying the product related to the disease developed to the user, a disease name, a disease-variation ID, and whether the disease has developed, and
wherein the selecting comprises:
if the selection is not the first selection, increasing a weight value to disease-variations related to the developed disease confirmed through the user feedback information, and reselecting the disease-variation to be used in predicting disease risk based on the weight value.

8. The method of claim 7, wherein the weight value is calculated based on whether the disease has developed and a number of variations discovered.

9. The method of claim 7, wherein the predicting disease risk comprises:
generating a user variation ID list by matching the genes and disease-variations related to the first-selected or reselected disease with the user gene information;
if the disease is a complex disease and the disease-variations comprised in the user variation ID list are not comprised in the product, determining the variations as not related to the disease and excluding the variations accordingly;
if the disease is a complex disease and the disease-variations comprised in the user variation ID list are comprised in the product, determining the variations as related to the disease and predicting disease risk based on the disease-variations comprised in the product;
if the disease is a rare disease and the disease-variations comprised in the user variation ID list are comprised in the product, classifying disease risk as high risk;
if the disease is a rare disease and the disease-variations comprised in the user variation ID list are not comprised in the product, but the disease-variations affect protein structures or cause loss of functions, classifying the subject disease as a high risk group; and
if the disease is a rare disease and the disease-variations comprised in the user variation ID list are not comprised in the product, or the disease-variations do not affect protein structures or cause loss of functions, determining the variations as not related to the disease and excluding the variations accordingly.

10. The method of claim 9, wherein the providing comprises providing a result report comprising a product version ID, a disease name, a variant ID, and a disease risk as mobile services through a smart phone application.

11. An apparatus for computer-based disease risk analysis connected to a network, comprising:
a disease-variation selecting data-base (DB) configured to store a disease-variation table, wherein the disease-variation table is a reference information table to set medical ground levels and disease-variation information to use in predicting disease risk;
a disease-variation selecting unit configured to select disease-variations related to diseases using the reference information table and includes the selected disease-variations information to the disease-variation table;
a disease risk predicting unit configured to predict a disease risk using the disease-variations in the disease-variation table;
a providing unit configured to provide results of disease risk predicted by the disease risk predicting unit to a user terminal through the network;
a user feedback unit configured to receive a feedback as to whether a disease has developed in a user from the user terminal; and
a weight value setting unit configured to confirm whether a disease has developed through the feedback and sets a weight value to at least one disease-variation used in predicting the risk of the developed disease wherein the disease-variation selecting unit is configured to select one having a relatively high weight value among the disease-variations comprised in the disease-variation table.

12. The apparatus of claim 11, wherein the reference information table comprises:
medical ground levels, which are a criteria representing an extent of strength with respect to a disease-variation correlation set based on ground levels reported in other disease-related DB, which represents cases where information in other disease DB that include information on number of samples used in disease-variation correlation studies, animal experiment certificates that represent cases where studies on genetic functions are performed through animal experiments, and statistical significance of disease-variation correlation studies and disease-variation correlation;
wherein the disease-variation selecting unit examines disease-related genes and variations from a plurality of websites and databases in which information on disease-related genes and mutations is stored, further examines research articles with respect to a correlation between diseases and race, and collects review information by experts, and selects disease-variations related to diseases based on the collected information and the medical ground levels.

13. The apparatus of claim 12, wherein
the disease-variation table
stores an ID and a version information of a product consisting of a combination of mutually-different diseases-variations, disease names, ID of disease-variations related to diseases, a medical ground level for each of the disease-variations, a weight value for each of the disease-variations, number of cases where the subject disease-variation are actually found among people who have used the product, a number that the product is offered, and a final relevance score calculated using the number of people in whom the disease was actually developed and the weight value; and
the disease-variation selecting unit selects disease-variations in an order from a highest relevance score to a lowest relevance score.

14. The apparatus of claim 13, wherein
the user feedback unit receives user feedback information, wherein the ID and version information of the product related to the disease developed in a user, a name of the disease, IDs of the disease-variations, and whether the disease has developed; and
the weight value setting unit increases weight values for the disease-variations related to the developed disease as confirmed through a user feedback information.

15. The apparatus of claim 14, wherein the weight value setting unit sets the weight values that are calculated on whether the disease has developed and number of variations discovered to the disease-variations.
